(19) United States
(12) Patent Application Publication
(10) Pub. No.: US 2008/0081028 A1
(43) Pub. Date: Apr. 3, 2008

(54) Pressed powder composition

(75) Inventor: Kojo Tanaka, Ashiya (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/904,531

(22) Filed: Sep. 27, 2007

Related U.S. Application Data
(60) Provisional application No. 60/847,781, filed on Sep. 28, 2006.
(60) Provisional application No. 60/874,532, filed on Dec. 12, 2006.

(51) Int. Cl. A61K 8/96; A61Q 1/02; A61Q 1/12

(52) U.S. Cl. 424/63

(57) ABSTRACT

Disclosed is a pressed powder composition having comprising by weight: (a) from about 75% to about 99% of a powder comprising: (1) at least 25% by weight of the entire composition of a high coverage pigment selected from the group consisting of titanium dioxide, zinc oxide, and mixtures thereof, (2) a natural mica selected from the group consisting of muscovite, phlogopite, mixtures thereof, and complexes thereof; the natural mica having a Natural Mica Percentage to the entire composition; (b) from about 1% to about 25% of a binder selected from the group consisting of non-volatile oil, lipophilic surfactant, humectant, solid fatty compounds, solid wax, solid gelling agents, solid silicone elastomers, and mixtures thereof; wherein the composition has a Product Oil Absorbency of from about 10 g/100 g to about 23 g/100 g, wherein the Product Oil Absorbency is the oil absorbency measured by JIS K5101 modified by: i) using the entire composition as a test sample; ii) using the binder as the oil to be absorbed; and iii) the unit transferred into g/100 g; wherein the composition has a Compaction Index of from about 25 to about 70, wherein the Compaction Index is described by the following formula: Compaction Index=2.69×Product Oil Absorbency+1.46×Natural Mica Percentage−31.44.

### CROSS REFERENCE TO RELATED APPLICATIONS

This application claims the benefit of U.S. Provisional Application No. 60/847,781 filed on Sep. 28, 2006; and U.S. Provisional Application No. 60/874,532 filed on Dec. 12, 2006.

### FIELD OF THE INVENTION

The present invention relates to a pressed powder composition which provides good coverage to skin imperfections, and also has appropriate cake hardness. The composition is particularly useful as a powder concealer. The present invention further relates to methods of make-up using such composition.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Concealers are used to obtain high coverage on a particularly concerned area, and supplement the function of a foundation on. Concealers are typically liquid, paste or semi-solid form products containing a high level of pigments having opacity, such as titanium dioxide, and are typically used prior to applying the foundation.

One of the unsatisfying points for concealer users is that, the portion of skin for which the concealer is applied provides an unnatural appearance. Namely, in view of different texture or different color provided by the concealer, the skin area on which concealer is applied is noticeable. Considering that concealers are used in desire to make a certain concerned area of the skin to be less conspicuous from the surrounding area, such unnatural appearance is contrary to what is desired.

Another unsatisfying point for concealer users is that, concealer products are typically in liquid form and are designed to apply to the skin prior to application of foundation. A concealer product in the powder form would be convenient to carry, and convenient to use as touch-up during the day. One of the difficulties for providing a concealer in the powder form, is that formulations containing high levels of titanium dioxide result in a hard cake when pressed. When cake hardness is too hard, the product is difficult to pick up with a finger or applicator, and the pay off of the product is significantly decreased.

Concealers are disclosed in, for example, Japanese Patent Publications 2000-327532, 2004-008307, 2005-298482, 6-56628, and 2003-277217. There are no references, however, which disclose concealers in the powder form that provide satisfactory cake hardness.

Based on the foregoing, there is a need for a pressed powder composition which provides improved natural coverage to skin imperfections, and also has appropriate cake hardness. None of the existing art provides all of the advantages and benefits of the present invention.

### SUMMARY

The present invention is directed to a pressed powder composition comprising by weight:
(a) from about 75% to about 99% of a powder comprising:
   (1) at least 25% by weight of the entire composition of a high coverage pigment selected from the group consisting of titanium dioxide, zinc oxide, and mixtures thereof,
   (2) a natural mica selected from the group consisting of muscovite, phlogopite, mixtures thereof, and complexes thereof; the natural mica having a Natural Mica Percentage to the entire composition;
(b) from about 1% to about 25% of a binder selected from the group consisting of non-volatile oil, lipophilic surfactant, humectant, solid fatty compounds, solid wax, solid gelling agents, solid silicone elastomers, and mixtures thereof;
   wherein the composition has a Product Oil Absorbency of from about 10 g/100 g to about 23 g/100 g, wherein the Product Oil Absorbency is the oil absorbency measured by JIS K5101 modified by:
   i) using the entire composition as a test sample;
   ii) using the binder as the oil to be absorbed; and
   iii) the unit transferred into g/100 g;
   wherein the composition has a Compaction Index of from about 25 to about 70, wherein the Compaction Index is described by the following formula:

Compaction Index=2.69×Product Oil Absorbency+1.46×Natural Mica Percentage−31.44.

The present invention is also directed to a pressed powder composition comprising by weight:
(a) from about 84% to about 94% of a powder comprising:
   (1) from about 25% to about 50% by weight of the entire composition of a high coverage pigment selected from the group consisting of titanium dioxide, zinc oxide, and mixtures thereof;
   (2) from about 10% to about 35% of a natural mica selected from the group consisting of muscovite, phlogopite, mixtures thereof, and complexes thereof;
(b) from about 6% to about 16% of a binder selected from the group consisting of non-volatile oil, lipophilic surfactant, humectant, solid fatty compounds, solid wax, solid gelling agents, solid silicone elastomers, and mixtures thereof;
   wherein the composition has a Product Oil Absorbency of from about 10 g/100 g to about 23 g/100 g, wherein the Product Oil Absorbency is the oil absorbency measured by JIS K5101 modified by:
   i) using the entire composition as a test sample;
   ii) using the binder as the oil to be absorbed; and
   iii) the unit transferred into g/100 g.

The present invention is further directed to a method of making up the skin utilizing the aforementioned composition as a concealer.

These and other features, aspects, and advantages of the present invention will become evident to those skilled in the art from a reading of the present disclosure with the appended claims.

### DETAILED DESCRIPTION OF THE INVENTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Composition and Method of Use

The present composition comprises from about 75% to about 99% of powder, and is in the form of a pressed powder. By pressed powder, what is meant is that the composition is pressed into a pan by a pressing machine commonly used in the art with a pressure of from about 3.0 MPa to about 7.0 MPa to form a cake having appropriate hardness and integrity. These product forms are widely used in the industry.

The present composition comprises at least about 25% by weight of the entire composition of high coverage pigment. The high amount of high coverage pigment provides such high coverage, and thus the present composition is particularly suitable for use as a concealer. Herein, a concealer is a composition which provides higher coverage than a foundation, and is typically used to a particular area of the skin for which specific care of appearance is desired.

In one preferred embodiment, the present composition is a concealer that is used with a foundation of the same product form, namely a pressed powder foundation. Without being bound by theory, it is believed that, by matching the product form of the foundation and concealer, the texture of the two compositions are very similar when applied to the skin, such that the concealer provides natural coverage to the portion of the skin where higher coverage is needed.

Preferably, the present composition has a color by comprising colored powders as described hereinbelow, dyes, and other colorant materials. In the preferred embodiment described above, the CMC DE of the concealer color is less than 1.5 compared to the foundation color with which the concealer is used. Accordingly, there is substantially no difference in color of the foundation and concealer in the eyes of the general consumer. The matching of the color of the foundation and concealer may be provided by 1) indication of color code on independently packaged foundation and concealer, 2) providing the foundation and concealer as a kit, or 3) providing the foundation and concealer in the same package.

In one preferred embodiment, the foundation and concealer have substantially no difference in color, and are of the same product form. By having such common color and product form, the combined use of the foundation and concealer provides a seamless appearance to the skin, namely, the portion where concealer were applied is not recognizable.

In one preferred embodiment, the foundation and concealer are provided in the same package. The package is a compact housing a pan for the foundation, a pan for the concealer, and an applicator. Preferably, two applicators are contained in the package such that the foundation and concealer can be used via separate applicators. Suitable for foundation is a sponge for broad application, and suitable for concealer is a tip for focused application.

The present invention relates to the method of making up the skin using the present composition as a concealer. Conventionally, concealers are recommended for use prior to application of the foundation. The present concealer may be used either prior to or after application of the foundation. For providing the concealer suitable for “after application” the concealer is preferably substantially the same color as the foundation. By providing such color the present concealer can be used as a touch up during the day.

### Natural Mica

The present composition comprises a natural mica, wherein the percentage of the natural mica to the entire composition is defined as “Natural Mica Percentage”. Preferably the natural mica is from about 1% to about 45%, more preferably from about 8% to about 45%, still preferably from about 10% to about 35% of the composition. It is possible, however, to provide compositions of appropriate cake hardness which have a natural mica percentage outside this preferable range, so long as the Compaction Index as explained below is met.

The natural micas useful herein are any that are obtained as natural minerals such as muscovite, phlogopite, and mixtures thereof, however, sericite and synthetic mica are not considered natural mica. The natural mica herein may be complexed via coating with a surface treatment agent or another type of pigment. When the natural mica is coated or complexed, only the weight of the natural mica is counted for the “Natural Mica Percentage”. Without being bound by theory, it is believed that natural mica, in combination with the other requisite powders, provide a soft yet appropriate cake hardness.

Commercially available natural mica materials that are highly preferred herein include mica coated with 5% aluminum dimyristate with tradename 5MI-MICA M-102 and mica coated with 2% methicone with tradename SI MICA, both available from Miyoshi Kasei.

### High Coverage Pigment

The present composition comprises at least about 25% by weight of the entire composition of a high coverage pigment. Preferably, the amount of high coverage pigment is from about 25% to about 60%, more preferably from about 25% to about 50% of the entire composition.

The high coverage pigment herein is selected from the group consisting of titanium dioxide, zinc oxide, and mixtures thereof, and have an average particle size of from about 100 nm to about 500 nm, preferably from about 200 nm to about 350 nm. The titanium dioxide may be rutile type or anatase type. The high coverage pigment is preferably at least partially used as complex pigments with other organic/inorganic components, and are also preferably hydrophobically coated. For calculating the amount of high coverage pigment of the complex pigments, only the high coverage pigment content or coating is counted.

Useful complexed pigments for the concealer include titanium coated pigment made of a core pigment selected from the group consisting of talc, mica, sericite, synthetic mica, aluminum oxide, silica, boron nitride, and mixtures thereof, which core pigment is coated with titanium dioxide having a particle size of from about 100 nm to about 500 nm, and the titanium dioxide coating accounting for at least 33% of the titanium coated pigment, preferably at least 40% of the titanium coated pigment. By such higher coating, such titanium coated pigments provide a high coverage effect compared to the same weight of pigments made solely of titanium dioxide.

Commercially available high coverage pigments highly useful herein include 57.3% titanium dioxide coated sericite further coated with methicone in the tradename of Fancyveil S-3060SW available from Shokubai Kasei, and Titanium Dioxide coated with Methicone with tradename SI Titanium Dioxide IS available from Miyoshi Kasei.

### Other Powders

The present composition contains powders other than the high coverage pigment and natural mica to provide color or change skin tone, or to provide other appearance and skin feel effects. In the preferred embodiment where the present composition has color, the other powders are used to provide a color resembling the skin, more preferably to have a color substantially the same color as the foundation to be used with. The total powder content is from about 75% to about 99%, preferably from about 84% to about 94% of the composition.

Useful other powders herein include clay mineral powders such as talc, sericite, bentonite and montmorillonite; coloring powders useful herein include iron oxides, iron titate, ultramarine blue, Prussian blue, chromium oxide, chromium hydroxide, cobalt oxide, cobalt titanate, laked tar color dyes, and laked natural color dyes; organic powders herein include polyacrylates such as methyl methacrylate copolymer and methyl methacrylate crosspolymers, celluloses, polyalkylenes such as polyethylene and polypropylene, vinyl acetates, polystyrenes such as styrene-acrylic acid copolymers, polyamides such as 12-nylon and 6-nylon, acrylic acid ethers such as acrylic acid methyl ether and acrylic acid ethyl ether, polyvinyl pyrrolidones; vinyl chloride polymers, silicones such as polyorganosilsesquioxane resin and solid silicone elastomers, tetrafluoroethylene polymer, and fish scale guanine; inorganic powders such as barium sulfate, calcium secondary phosphate, hydroxy apatite, silicates; such as calcium silicate, magnesium silicate, barium silicate and aluminium silicate, silica beads, metal dioxides such as zirconium oxide and aluminium hydroxide, carbonates such as calcium carbonate and magnesium carbonate, boron nitride and synthetic fluorphlogopite. UV shielding metal oxides such as titanium dioxide and zinc oxide having an average particle size of less than about 100 nm are also useful herein.

Polyorganosilsesquioxane resin and solid silicone elastomers may be used for enhancing the effect of hiding skin pores.

In one preferred embodiment, the composition may comprise a metal soap coated pigment made of a core pigment selected from the group consisting of sericite, talc, synthetic mica, aluminum oxide, silica, boron nitride, and mixtures thereof, which core pigment is coated with one or more metal soap, such as aluminum dimyristate, aluminium stearate, magnesium stearate, zinc myristate, magnesium myristate, zinc palmitate, zinc laurate, calcium stearate, and mixtures thereof, the metal soap coating accounting for at least 1% of the metal soap coated pigment, preferably at least 3% of the metal soap coated pigment. By such higher coating, such metal soap coated pigment provides good adhesion between itself and the skin, as well as between the pigments themselves.

The powders herein may be surface coated with a coating material having hydrophobic characteristics, or lipophobic hydrophobic characteristics. Useful hydrophobic coating materials herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof. Useful lipophobic hydrophobic coating materials are fluorine compounds such as perfluorooctyl triethoxylsilane, perfluoroalkylphosphoric acids, their salts, and mixtures thereof.

Commercially available powders highly useful herein include methyl methacylate crosspolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, vinyl dimethicone/methicone silsesquioxane crosspolymer with tradenames KSP series available from ShinEtsu Chemical Co., Ltd., Tokyo Japan, hardened polyorgano siloxane elastomers with tradenames TREFIL series available from Toray Dow Corning, boron nitride with tradename SHP series available from Mizushima Ferroalloy Co., Ltd., 5% aluminum dimyristate coated sericite in the name of 5MI-SERICITE, and 5% aluminum dimyristate coated talc in the name of 5MI-TALC JA-46R, all available from Miyoshi Kasei.

### Binder

The present composition comprises from about 1% to about 25%, preferably from about 6% to about 16% of a binder selected from the group consisting of non-volatile oil, lipophilic surfactant, humectant, solid fatty compounds, solid wax, solid gelling agents, solid silicone elastomers, and mixtures thereof. The solid species herein may be useful at low levels, however, such components are kept to no more than 5% of the entire composition, and no more than 40% of the entire binder component.

Useful for the composition of the present invention is a non-volatile oil. The non-volatile oil herein is believed to provide improved smoothness to the skin, and also alleviate dry feeling of the skin.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Liquid UV absorbing agents such as ethylhexyl methoxycinnamate are also useful as non-volatile oils.

Commercially available oils include, for example, tridecyl isononanoate with tradename Crodamol TN available from Croda, Hexylan available from Nisshin Seiyu, and tocopherol acetates available from Eisai.

Non-volatile oils useful herein also include polyalkyl or polyaryl siloxanes with the following structure (I)

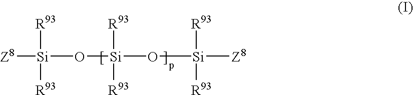

wherein R93 is alkyl or aryl, and p is an integer from about 7 to about 8,000. Z8 represents groups which block the ends of the silicone chains. The alkyl or aryl groups substituted on the siloxane chain (R93) or at the ends of the siloxane chains Z8 can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z8 groups include hydroxy, methyl, methoxy, ethoxy, propoxy, and aryloxy. The two R93 groups on the silicon atom may represent the same group or different groups. Preferably, the two R93 groups represent the same group. Suitable R93 groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Useful for the composition of the present invention is a lipophilic surfactant. The lipophilic surfactant herein has an HLB value of less than about 8.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), (iii), and (iv) as shown below, and mixtures thereof.

(i) dimethicone copolyols having the formulation:

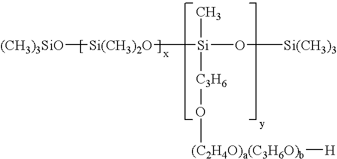

wherein x is an integer from 5 to 100, y is an integer from 1 to 50, a is zero or greater, b is zero or greater, the average sum of a+b being 1-100.
(ii) dimethicone copolyols having the formulation:

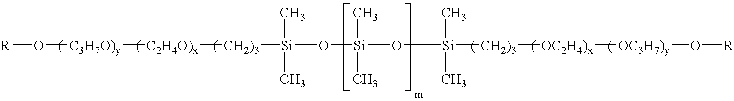

wherein R is selected from the group consisting of hydrogen, methyl, and combinations thereof, m is an integer from 5 to 100, x is independently zero or greater, y is independently zero or greater, the sum of x+y being 1-100.
(iii) branched polyether-polydiorganosiloxane emulsifiers herein having the formulation:

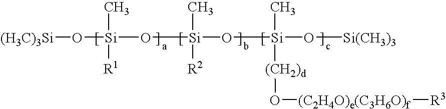

wherein R1 is an alkyl group having from about 1 to about 20 carbons; R2 is

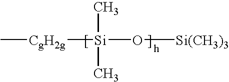

wherein g is from about 1 to about 5, and h is from about 5 to about 20; R3 is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.
(iv) alkyl dimethicone copolyols which are nonionic polysiloxane copolymer having emulsifying ability, comprising a methylpolysiloxane moiety, an alkyl methylpolysiloxane moiety, and a poly(oxyalkylene)methylpolysiloxane moiety; having an HLB from about 4 to about 6, and a molecular weight of from about 10,000 to about 20,000, wherein the alkyl group is made of from about 10 to about 22 carbons. Suitable alkyl dimethicone copolyols herein are those which have the following formulation:

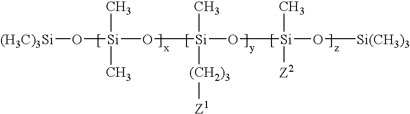

wherein Z1 is O(C2H4O)p(C3H6O)qH, p is from 0 to about 50, q is from 0 to about 30, wherein p and q are not 0 at the same time; x is from 1 to about 200, y is from 1 to about 40, and z is from 1 to about 100, and Z2 is an alkyl group having from about 10 to about 22 carbons, preferably from about 16 to about 18 carbons.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF 6028 available from ShinEtsu Chemical. Highly preferred alkyl dimethicone copolyols include cetyl dimethicone copolyol and stearyl dimethicone copolyol. A highly preferred commercially available alkyl dimethicone copolyol includes cetyl dimethicone copolyol, also called Methylpolysiloxane Cetylmethylpolysiloxane Poly(oxyethylene oxypropylene) Methylpolysiloxane Copolymer, having an HLB of about 5 and a molecular weight of about 13,000 having a tradename ABIL EM90 available from Goldschmidt Personal Care.

Useful for the composition of the present invention is a humectant.

The humectants herein are selected from the group consisting of polyhydric alcohols, water soluble alkoxylated nonionic polymers, and mixtures thereof. Polyhydric alcohols useful herein include glycerin, propylene glycol, 1,3-butylene glycol, dipropylene glycol, diglycerin, sodium hyaluronate, and mixtures thereof.

Commercially available humectants herein include: glycerin available from Asahi Denka; propylene glycol with tradename LEXOL PG-865/855 available from Inolex, 1,2-PROPYLENE GLYCOL USP available from BASF; 1,3-butylene glycol available from Kyowa Hakko Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

Useful for the composition of the present invention is a solid fatty compound.

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

Useful for the composition of the present invention is a solid wax.

The solid waxes useful herein are paraffin wax, microcrystalline wax, ozokerite wax, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

Useful for the composition of the present invention is a solid gelling agent.

The gelling agents useful herein include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:

R2-NH—CO—(CH₂)2-CH—(NH—CO—R1)—CO—NH—R2

wherein R1 is an aliphatic hydrocarbon radical having from about 12 to about 22 carbon atoms, and R2 is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercially available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:

R1-CO—(CH₂)10-CH—(OH)—(CH2)5-CH3

wherein R1 is R2 or NR2R3; and R2 and R3 are hydrogen, or an alkyl, aryl, or arylalkyl radical which is branched linear or cyclic and has from about 1 to about 22 carbon atoms; preferably, from about 1 to about 18 carbon atoms. R2 and R3 may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Useful for the composition of the present invention is a solid silicone elastomer.

Suitable for use herein are silicone elastomers which can be emulsifying or non-emulsifying crosslinked siloxane elastomers or mixtures thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. Nos. 4,970,252, 5,760,116, and 5,654,362. Additional crosslinked organopolysiloxane elastomers useful in the present invention are disclosed in Japanese Patent Application JP 61-18708, assigned to Pola Kasei Kogyo KK. Commercially available elastomers preferred for use herein are Dow Corning's 9040 silicone elastomer blend, Shin Etsu's KSG-21, and mixtures thereof.

### Product Oil Absorbency

The present composition has a Product Oil Absorbency which is measured by JIS K5101 modified by:

i) using the entire composition as a test sample;
ii) using the binder as the oil to be absorbed; and
iii) the unit transferred into g/100 g.

The Product Oil Absorbency describes the oil absorbing capability of the pressed powder composition herein in its entirety, rather than only its powder component. Further, in that the Product Oil Absorbency is an indicator for aiding formulation design, the oil used for measurement is the binder components of the pressed powder composition. Namely, the Product Oil Absorbency describes the oil absorbency capability of the powder composition per se, holistically taking into consideration the amount and species of the powder and binder of the composition. It is well known that the type and amount of high oil absorbing powders in the formulation may affect oil absorbency, however, Product Oil Absorbency also takes into consideration the interaction of such powders with the binder.

The present composition has a Product Oil Absorbency of from about 10 g/100 g to about 23 g/100 g, more preferably from about 13 g/100 g to about 21 g/100 g. While it is possible to provide compositions of appropriate cake hardness outside this range, Product Oil Absorbency is controlled to from about 10 g/100 g to about 23 g/100 g to avoid too dry or too wet feel to the skin.

### Measurement of Product Oil Absorbency

The quantity of binder (used in the entire composition) to be absorbed in the entire composition (hereinafter “sample”) under specified condition is measured, and Product Oil Absorbency is obtained.

### 1. Preparation

Following apparatus are prepared:
a) A glass plate 300×400×5 mm
b) A stainless steel knife (edge length: 150 mm, edge width 33 mm)
c) A chemical balance capable of measuring to the nearest 10 mg.
d) A 50 ml glass beaker
e) A stirrer capable to heat and a stirrer piece
f) A small plastic dropper
g) A charta and a spatula

### 2. Materials

a) Binder—The binder component only of the entire composition is prepared, with heat added in case solid components are included. A portion of the binder is transferred to a 50 ml beaker, and a stirrer piece and a dropper are put and gross weight is weighed by a chemical balance. Then after removing the dropper, they are put on a stirrer and mixed slowly to keep it homogenous. In case solid oils are included, during stirring, heating is also required.
b) Sample—The entire composition is separately obtained and used as sample. A charta was put on the chemical balance and 4.0 g of the sample is weighed by a spatula.

### 3. Operation

a) Place 4.0 g of the sample in the center of glass plate, using a dropper, gradually drop each 1-4 drops per once of binder onto the center of the sample, and sufficiently kneaded the sample with the palette knife each time.
b) Repeat the dropping and kneading procedure until the sample becomes one lump of hard putty like lump. This is the end point. Drop rate is adjusted such that this procedure is this completed within 7 to 15 min.
c) Weigh the gross weight of the beaker, the stirrer piece, used binder and the dropper, then calculate how much binder is added when the end point is reached.

### 4. Calculation

### The Product Oil Absorption is calculated from the following formula to be rounded off to one decimal place.

Product Oil Absorption=W/4.0×100 (g/100 g)

wherein W: weight of added the oil binder (g)

### Compaction Index

The present composition has a Compaction Index of from about 25 to about 70, preferably from about 35 to about 60, wherein the Compaction Index is described by the following formula:

Compaction Index=2.69×Product Oil Absorbency+1.46×Natural Mica Percentage−31.44.

The Compaction Index herein is a parameter for determining the appropriate cake hardness of the composition when pressed. Cake hardness is an important factor for a pressed powder form product. If the cake hardness is too hard, the product is difficult to pick up with a finger or applicator, and the pay off of the product is significantly decreased. If the cake hardness is too soft, the cake is so brittle that the cake is easily cracked during transportation or carrying by the user. The Compaction Index provides a way for one skilled in the art to formulate a pressed powder composition having appropriate cake hardness over a wide range of powders and binders that may be used for the composition. Herein, the oil absorbency of the entire composition, rather than just the powder, is measured, as defined under Product Oil Absorbency above.

The Compaction Index formula defines the balance of the Product Oil Absorbency and the amount of natural mica to include in the composition. When the Product Oil Absorbency is relatively low, the amount of mica may be increased to provide an appropriate Compaction Index. On the other hand, when the Product Oil Absorbency is relatively high, the amount of mica may be decreased to provide an appropriate Compaction Index. However, not all compositions having Product Oil Absorbency and Natural Mica Percentage in its preferred ranges provide an acceptable Compaction Index. Such compositions having a Compaction Index outside the defined range are less preferred.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Composition for Examples 1-7

| | | Ex. 1 | Ex. 2 | Ex. 3 | Ex. 4 | Ex. 5 | Ex. 6 | Ex. 7 |
|---|---|---|---|---|---|---|---|---|
| 1 | Talc coated with 5% Aluminum | 5.74 | 8.24 | 1.24 | 21.24 | 0.24 | 23.24 | |
| | Dimyristate *1 | | | | | | | |
| 2 | Sericite coated with 5% | 23 | 23 | 15 | 23 | | 23 | |
| | Aluminum Dimyristate *2 | | | | | | | |
| 3 | Mica coated with 5% Aluminum | 17.5 | 10 | 30 | 5 | 45 | 5 | 29.15 |
| | Dimyristate *3 | | | | | | | |
| 4 | Sericite coated with 57.3% | 5 | 5 | 5 | 5 | 5 | 5 | 50 |
| | Titanium Dioxide and Methicone *4 | | | | | | | |
| 5 | Titanium Dioxide coated with | 27 | 27 | 27 | 27 | 27 | 27 | |
| | Methicone *5 | | | | | | | |
| 6 | Methyl Methacrylate | 8 | 8 | 8 | 8 | | 8 | |
| | Crosspolymer *6 | | | | | | | |
| 7 | Niacinamide *7 | 0.1 | 0.1 | 0.1 | 0.1 | 0.1 | 0.1 | 0.1 |
| 8 | Panthenol *8 | 0.1 | 0.1 | 0.1 | 0.1 | 0.1 | 0.1 | 0.1 |
| 9 | Yellow Iron Oxide coated with | 3.84 | 3.84 | 3.84 | 3.84 | 3.84 | 3.84 | 5.5 |
| | Methicone *9 | | | | | | | |
| 10 | Black Iron Oxide coated with | 0.4 | 0.4 | 0.4 | 0.4 | 0.4 | 0.4 | 0.48 |
| | Methicone *10 | | | | | | | |
| 11 | Red Iron Oxide coated with | 0.82 | 0.82 | 0.82 | 0.82 | 0.82 | 0.82 | 1.17 |
| | Methicone *11 | | | | | | | |
| 12 | Dimethicone *12 | 3.85 | 8.85 | 3.85 | 0.85 | 12.85 | 0 | 8.85 |
| 13 | Ethylhexyl Methoxycinnamate *13 | 4 | 4 | 4 | 4 | 4 | 2.85 | 4 |
| 14 | D-delta-tocopherol *14 | 0.1 | 0.1 | 0.1 | 0.1 | 0.1 | 0.1 | 0.1 |
| 15 | Perfume | 0.05 | 0.05 | 0.05 | 0.05 | 0.05 | 0.05 | 0.05 |
| 16 | Propyl paraben | 0.2 | 0.2 | 0.2 | 0.2 | 0.2 | 0.2 | 0.2 |
| 17 | Methyl paraben | 0.3 | 0.3 | 0.3 | 0.3 | 0.3 | 0.3 | 0.3 |
| | Total | 100 | 100 | 100 | 100 | 100 | 100 | 100 |

| | | | | | | | |
|---|---|---|---|---|---|---|---|
| Product Oil Absorbency (g/100 g) | 20.0 | 13.0 | 23.0 | 22.0 | 13.8 | 25.7 | 17.0 |
| Natural Mica Percentage (wt %) | 16.6 | 9.5 | 28.5 | 4.8 | 42.8 | 4.8 | 27.7 |
| Compaction Index | 46.6 | 17.4 | 72 | 34.7 | 68.2 | 44.7 | 54.7 |

Definitions of Components
*1 Talc coated with 5% Aluminum Dimyristate: 5MI-TALC JA-46R available from Miyoshi Kasei
*2 Sericite coated with 5% Aluminum Dimyristate: 5MI-SERICITE available from Miyoshi Kasei
*3 Mica coated with 5% Aluminum Dimyristate: 5MI-MICA M-102 available from Miyoshi Kasei
*4 Sericite coated with 57.3% Titanium Dioxide and Methicone: Fancyveil S-3060SW available from Shokubai Kasei
*5 Titanium Dioxide coated with Methicone: SI Titanium Dioxide IS available from Miyoshi Kasei
*6 Methyl Methacrylate Crosspolymer: GANZ PEARL GMX-0610 available from GANZ CHEMICAL CO.
*7 Niacinamide: Niacinamide available from Reilly Industries Inc
*8 Panthenol: DL-Panthenol available from Alps Pharmaceutical Inc
*9 Yellow Iron Oxide coated with Methicone: SI MAPICO YW LIGHT LEMON XLO available from Daito Kasei
*10 Black Iron Oxide coated with Methicone: SI BLACK IRON OXIDE NO. 247 available from Daito Kasei
*11 Red Iron Oxide coated with Methicone: SI PURE RED IRON OXIDE R-3098 available from Daito Kasei
*12 Dimethicone: SILICONE OIL SH200C-50CS available from Dow Corning
*13 Ethylhexyl Methoxycinnamate: PARSOL MCX available from ROCHE VITAMINS JAPAN K.K
*14 D-delta-tocopherol: D-DELTA-TOCOPHEROL available from EISAI CO., LTD.

### Method of Preparation

Component numbers 1-11 and 17 were mixed with a mixer to make a powder component. Separately, component numbers 12-16 were mixed to make a binder component. The binder component was added into the powder component and mixed by a mixer. The obtained composition was distributed at 10.5 g per pan and pressed at 5.0 MPa.

### Measurement of Product Oil Absorbency

The binder was prepared by mixing component numbers 12-16 of each Example. For each measurement, 4.0 g sample of the sample of Examples 1-7, respectively, were placed on a glass plate. For each Example, 1-4 drops of the respective binder were dropped on the sample and kneaded by a pallet knife each time. The amount of the added binder was measured. The Product Oil Absorbency of each Example was calculated as described above.

### Calculation of Natural Mica Percentage and Compaction Index

The Natural Mica Percentage of each Example was obtained by calculating the natural mica percentage of component number 3. The Compaction Index of each Example was obtained by entering the Product Oil Absorbency and Natural Mica Percentage into the following formula. Compaction Index=2.69×Product Oil Absorbency+1.46×Natural Mica Percentage−31.44

### Evaluation

Example 2, having a Compaction Index of less than 25, had a too hard cake hardness, that pay off of the product using a buff was too small. Example 3, having a Compaction Index of more than 70, had a too soft cake hardness, that the product was easily cracked by dropping from a height of 30 cm. Example 6, having a Product Oil Absorbency of over 23, provided a dry feeling upon application to the skin. The remaining Examples 1, 4, 5, and 7 provided good pay off using a buff, had appropriate tolerance to cracking when dropped from a height of 30 cm, and provided good skin feel when applied on the skin.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A pressed powder composition comprising by weight:
(a) from about 75% to about 99% of a powder comprising:
(1) at least 25% by weight of the entire composition of a high coverage pigment selected from the group consisting of titanium dioxide, zinc oxide, and mixtures thereof,
(2) a natural mica selected from the group consisting of muscovite, phlogopite, mixtures thereof, and complexes thereof; the natural mica having a Natural Mica Percentage to the entire composition;
(b) from about 1% to about 25% of a binder selected from the group consisting of non-volatile oil, lipophilic surfactant, humectant, solid fatty compounds, solid wax, solid gelling agents, solid silicone elastomers, and mixtures thereof;
wherein the composition has a Product Oil Absorbency of from about 10 g/100 g to about 23 g/100 g, wherein the Product Oil Absorbency is the oil absorbency measured by JIS K5101 modified by:
i) using the entire composition as a test sample;
ii) using the binder as the oil to be absorbed; and
iii) the unit transferred into g/100 g;
wherein the composition has a Compaction Index of from about 25 to about 70, wherein the Compaction Index is described by the following formula:
Compaction Index=2.69×Product Oil Absorbency+1.46×Natural Mica Percentage−31.44.

2. The composition of claim 1 wherein the Natural Mica Percentage is from about 1% to about 45%.

3. The composition of claim 1 wherein the Compaction Index is from about 35 to about 60.

4. A pressed powder composition comprising by weight:
(a) from about 84% to about 94% of a powder comprising:
(1) from about 25% to about 50% by weight of the entire composition of a high coverage pigment selected from the group consisting of titanium dioxide, zinc oxide, and mixtures thereof;
(2) from about 10% to about 35% of a natural mica selected from the group consisting of muscovite, phlogopite, mixtures thereof, and complexes thereof;
(b) from about 6% to about 16% of a binder selected from the group consisting of non-volatile oil, lipophilic surfactant, humectant, solid fatty compounds, solid wax, solid gelling agents, solid silicone elastomers, and mixtures thereof;
wherein the composition has a Product Oil Absorbency of from about 10 g/100 g to about 23 g/100 g, wherein the Product Oil Absorbency is the oil absorbency measured by JIS K5101 modified by:
i) using the entire composition as a test sample;
ii) using the binder as the oil to be absorbed; and
iii) the unit transferred into g/100 g.

5. The composition of claim 4 wherein the powder comprises colored powders.

6. The composition of claim 5 which is a concealer to be used with a foundation, the foundation having a color, and wherein the CMC DE of the foundation color and the concealer color is less than 1.5.

7. A method of making up the skin comprising the steps of:
(1) providing a concealer and foundation according to claim 6;
(2) applying to the skin the foundation; and
(3) applying to the skin the concealer.
